(19) United States
(12) Patent Application Publication
(10) Pub. No.: US 2020/0010807 A1
(43) Pub. Date: Jan. 9, 2020

(54) STEPWISE METHOD FOR INDUCING CHOLANGIOCYTE PROGENITORS FROM HEPATOBLASTS

(71) Applicant: Kyoto University, Kyoto (JP)

(72) Inventors: Kenji OSAFUNE, Kyoto (JP); Satoshi MATSUI, Kyoto (JP)

(21) Appl. No.: 16/461,905

(22) PCT Filed: Nov. 21, 2017
(86) PCT No.: PCT/JP2017/041806

§ 371 Date: Sep. 16, 2019

(30) Foreign Application Priority Data
Nov. 22, 2016 (JP) 2016-227128

(51) Int. Cl. C12N 5/071

(52) U.S. Cl. CPC C12N 5/0679; C12N 2501/11; C12N 2501/12; C12N 2506/14; C12N 2501/42; C12N 2513/00; C12N 2506/45; C12N 2501/15

(57) ABSTRACT

Provided are: a method for producing biliary epithelial progenitor cells, said method comprising a step for culturing hepatoblasts in a medium containing TGFβ and EGF; and a method for constructing a three-dimensional duct-like structure of biliary epithelial progenitor cells, said method comprising a step for culturing hepatoblasts or biliary epithelial progenitor cells in a medium containing HGF, EGF, a Notch inhibitor and a GSK3 inhibitor in the presence of a three-dimensional scaffold material.

### TECHNICAL FIELD

This application relates to an in vitro stepwise method for inducing cholangiocyte progenitors from human hepatoblasts. Specifically, the present application provides a method for inducing cholangiocyte progenitors through the ductal plate stage to the remodeling ductal plate stage. This application also provides a method for generating a cell culture having a three-dimensional (3D) structure from cholangiocyte progenitors at the remodeling ductal plate stage.

### BACKGROUND TECHNOLOGY

The development of cholangiocytes begins with the formation of single layer of epithelial-like structure by hepatoblasts derived from the endoderm and foregut and adjacent to the periportal mesenchyme. This one-layer structure of cholangiocyte progenitors appears from around 8 weeks of gestation (gestation week: GW) and is called ductal plate (DP) stage. Subsequently, a bilayer structure is formed and between the layers, a duct is formed. In this way, the development of the bile duct having the duct structure proceeds. The stage in which this duct emerges is called remodeling ductal plate (RDP) stage, which corresponds to GW 12 and later. It has been known that cholangiocyte progenitors at the RDP stage start to express maturation markers such as AQP1 and CK7 (Non-Patent Literature 1). The biliary system development starts around the portal vein and proceeds efferently. Abnormality in the bile duct development is called ductal plate malformation (DPM).

Many diseases in which DPM is the initial phenotype and there is still no radical treatment has been known. Examples of these diseases include autosomal recessive polycystic kidney-disease (ARPKD), Alagille syndrome, and congenital biliary atresia. The development of a new therapy for those diseases has been desired.

However, no animal model that accurately mimics human conditions of those diseases is available, and gene expression pattern of human cholangiocyte lineages is different from that of rodents (Non-Patent Literature 2). It is not possible to accurately analyze human conditions by using rodent models. It is technically and ethically difficult to analyze human fetal samples. Accordingly, it has been difficult to analyze the early stages of various diseases associating with abnormalities in the development of the biliary system or to develop a therapy for those diseases.

Recently, disease models using iPS cells developed by Yamanaka et al., of Kyoto University (Non-Patent Literature 3) have been actively studied. The present inventors have established methods for inducing pancreatic bud cells, hepatocytes, and pancreatic hormone-producing cells from human iPS cells (Patent Literatures 1-3). For cholangiocyte progenitors, establishment of a method to induce differentiation of human iPS cells into endoderm ceils, hepatoblasts, ductal plate (DP) cells, remodeling ductal plate (RDP) cells, and mature cholangiocytes in a stepwise manner in accordance with their developmental process would be a good tool to analyze the disease phenotype in accordance with the developmental stage.

Organ-specific marker genes that are corresponding to albumin in hepatocytes or insulin in pancreatic β cells, have not been known for cholangiocytes. This fact has barred the development of methods to induce stepwise differentiation into cholangiocyte progenitors.

Recently, methods for inducing differentiation into cholangiocytes have begun to be reported, but the number is very small. Moreover, none of them mentions the stepwise induction of cholangiocyte progenitors. In Ogawa et al., (Non-Patent Literature 4), 3D culture of hepatoblasts was performed and final products were evaluated. However, it was not clear whether the final products were induced through the stages corresponding to the DP and RDP stages. Assancao et al., and Dianat et al., (Non-Patent Literatures 5 and 6) also did not address cholangiocyte progenitors. Only Sampaziotis et al., (Non-Patent Literature 7), mentioned the identification of cholangiocyte progenitors (CPs), but there was no description whether the progenitors were at the stage of DP or RDP.

### CITATION LIST

### Patent Literature

[Patent Literature 1]WO2015/020113

[Patent Literature 2]WO2015/178431

[Patent Literature 3]WO2016/104717

### Non-Patent Literature

[Non-Patent Literature 1]Vestentoft et al., BMC Developmental Biology 2011, 11:56.

[Non-Patent Literature 2]Glaser S et al., World J Gastroenterol. 2006 Jun 14;12 (22):3523-36.

[Non-Patent Literature 3]Takahashi, K et al, Cell 131, 861-872.

[Non-Patent Literature4 ]Ogawa M et al., Nat Biotechnol. 2015 Aug;33(8):853-61.

[Non-Patent Literature 5]De Assuncao TM et al., Lab Invest. 2015 Jun;95(6):684-96.

[Non-Patent Literature 6]Dianat N et al., Hematology. 2014 Aug;60(2):700-14.

[Non-Patent Literature 7]Sampaziotis F et al., Nat Biotechnol. 2015 Aug;33(8):845-52.

### SUMMARY OF INVENTION

### Problem to be Solved by Invention

An object of the present application is to provide a stepwise method for inducing cholangiocyte progenitors from hepatoblasts. Especially, an object of the present application is to provide a method for inducing cholangiocyte progenitors from pluripotent stem cells, such as iPS cells through hepatoblasts, to give ductal plate (DP)-like cholangiocyte progenitors and then, remodeling ductal plate (RDP)-like cholangiocyte progenitors.

Another object of the present application is to provide a method for generating a 3D duct-like structure from the cholangiocyte progenitors. The present application further provides a method for identifying a cholangiocyte progenitor that is corresponding to a cell at the RDP stage and a method of isolating said cell.

### Means to Solve the Problem

The present application provides a method for generating cholangiocyte progenitors, comprising the steps of providing hepatoblasts, and culturing the hepatoblasts in a medium comprising TGFβ and EGF. According to this method, DP-like and then, RDP-like cholangiocyte progenitors can be induced over time.

According to the present application, the hepatoblasts may be those induced from pluripotent stem cells. Various methods for inducing hepatoblasts from pluripotent stem cells have been reported and any known method may be employed.

The present application also provides a method for generating a 3D duct-like structure of cholangiocyte progenitors, comprising culturing hepatoblasts or cholangiocyte progenitors in a medium containing HGF, EGF, a Notch signal ligand and a GSK3 inhibitor in the presence of a 3D scaffold material.

The present application further provide a cell culture of cholangiocyte progenitors obtained by the method provided herein, and a 3D duct-like tissue wherein the cholangiocyte progenitors form a duct-like structure.

The present application further provides a method for confirming the maturity of a cholangiocyte progenitor and a method for isolating a cholangiocyte progenitor in accordance with the maturity based on the expression of AQP1 as an index.

By the present methods, it becomes possible to induce hepatoblasts into cholangiocyte progenitors in a stepwise manner. In addition, it becomes possible to form a 3D duct-like structure of cholangiocyte progenitors. The method provided herein will be useful for the elucidation of the mechanism of the differentiation of cholangiocyte progenitors from the DP stage to the RDP stage, the pathological analysis of diseases related to DPM, the developments of in vitro models of the diseases, and the developments of screening methods for candidate substances for the treatment of the diseases.

### BRIEF EXPLANATION OF DRAWINGS

FIG. 1 A schematic diagram of the AQP1-GFP construct used in the examples.

FIG. 2 A schematic diagram of the whole examples provided in this application.

FIG. 3 A schematic diagram of the induction of endoderm from human iPS cells. Expression of SOX17 in the induced cells is also shown. Hoechst stains the nuclei of the cells.

FIG. 4 A schematic diagram of the induction of hepatoblasts from the endoderm. Expressions of AFP and CK19 in the induced cells are also shown. Hoechst stains the nuclei of the cells.

FIG. 5-1 A schematic diagram of the process of inducing the cholangiocyte progenitors at the RDP stage from the hepatoblasts through the cholangiocyte progenitors at the DP stage.

FIG. 5-2 Photographs showing SOX9 and AQP1 expressions in the cells differentiated until Day 14. SOX9 was expressed extensively, but AQP1 was not expressed.

FIG. 5-3 Photographs showing SOX9 and AQPI expression in the cells differentiated until Day 18. AQP1 was expressed in a large parts of the cells.

FIG. 5-4 Photographs showing SOX9, CK19, ALB, AQP1 and CK7 expressions in the cells differentiated until Day 18. All of SOX9, CK19, ALB, AQP1 and CK7 were expressed.

FIG. 6-1 Cells differentiated until Day 18 were divided into GFP (+) that were AQP1-positive cells, and GFP (−) that were AQP1-negative cells, and gene expression profiles of each cell population were confirmed by PCR. Gene expression profiles of the cells obtained from fetal liver at gestation week 20 were also confirmed and served as control.

FIG. 6-2 Cells differentiated until Day 18 were divided into GFP (+) that were AQP1-positive cells, and GFP (−) that were AQP1-negative cells, and gene expression profiles of each cell population were confirmed by PCR. Gene expression profiles of the cells obtained from fetal liver at gestation week 20 were also confirmed and served as control.

FIG. 6-3 Cells differentiated until Day 18 were divided into GFP (+) that were AQP1-positive cells, and GFP (−) that were AQP1-negative cells, and gene expression profiles of each cell population were confirmed by PCR. Gene expression profiles of the cells obtained from fetal liver at gestation week 20 were also confirmed and served as control.

FIG. 7-1 Hepatoblasts on Day 11 and cholangiocyte progenitors on Day 14 were subjected to the 3D culture for 10 days, respectively. In both cases, duct-like structure of the cells expressing CK19 were observed. Scale bar indicates 50 μm.

FIG. 7-2 Results of rhodamine 123 uptake studies of the cell culture having the 3D duct-like structure derived from cholangiocyte progenitors on Day 14. Rhodamine was incorporated in the absence of verapamil. The scale bar indicates 50 μm.

### EMBODIMENTS FOR CONDUCTING THE INVENTION

According to the present application, hepatoblasts are cultured in a medium containing transforming growth factor (TGF)β and epithelial growth factor (EGF).

In this application, hepatoblasts are cells that have an ability to differentiate into hepatocytes and cholangiocytes. Human hepatoblasts are cells positive for at least one marker genes selected from the group consisting of AFP, CK19, Dlk, E-cadherin, Liv2, CD13 and CD133. Preferably, hepatoblasts are positive for AFP and CK19.

According to the method of the present application, hepatoblasts may be provided as a cell population comprising other cell types or may be as a purified population. Examples of methods for purifying hepatoblasts include staining the cells with antibodies directing to genetic markers such as AFP, CK19, Dlk, E-cadherin, Liv2, CD 13 or CD 133, and enriching the stained cells using a flow cytometer (FACS) or magnetic cell separator (MACS).

The medium used for differentiating hepatoblasts into cholangiocyte progenitors may be prepared by adding TGFβ and EGF to a basal medium as appropriate.

Examples of basal media include IMDM, Medium 199, Eagle's Minimum Essential Medium (EMEU), α-MEM, Dulbecco's modified Eagle's Medium (DMEM), Ham's F12 Medium, RPMI 1640 Medium, Fischer's Medium. Examples of basal media may also include HBM™ Basal Medium, HCM™ SingleQuots™ Kit, HMM™ Basal Medium and HMM™ SingleQuots™ Kit (Lonza), Hepatocyte Growth Medium and Hepatocyte Maintenance Medium (PromoCell), and Hepatocyte Medium (Sigma-Aldrich). A mixture of two or more of these media may also be used as the basal medium. The basal medium may be supplemented with serum, for example/ fetal bovine serum (FBS). Alternatively, a serum-free medium may be used. When a serum-free medium is used, as needed, the basal medium may contain, for example, one or more serum alternatives such as albumin, transferrin, Knockout™ Serum Replacement (KSR, a serum alternative used for culturing ES cells) (ThermoFisher Scientific), N2 Supplement (ThermoFisher Scientific), B27 Supplement (ThermoFisher Scientific), a fatty acid, insulin, sodium selenite, a collagen precursor, a trace element, 2-mercaptoethanol and 3′-thioglycerol. The basal medium may also contain one or more substances such as a lipid, an amino acid, L-glutamine, GlutaMAX (ThermoFisher Scientific), a nonessential amino acid (NEAA), a vitamin such as nicotinamide and ascorbic acid, a growth factor, an antibiotic, an antioxidant, pyruvic acid, a buffering agent, an inorganic salt, glucagon, hydrocortisone, dexamethasone and an equivalent thereof.

In this application, TGFβ may be any of TGFβ1, TGFβ2 and TGFβ3, and TGFβ2 may be preferable.

Commercially available TGFβ may be used. When TGFβ2 is employed as TGFβ, the concentration of TGFβ2 in the medium may be 1-100 ng/ml, preferably 2-50 ng/ml and for example, about 10 ng/ml.

Commercially available EGF (Epidermal growth factor) may be used. When human hepatoblasts are subjected to the differentiation, human EGF is preferably used. The concentration of EGF in the medium may be 2.5-250 ng/ml, preferably 5-125 ng/ml and for example, about 25 ng/ml.

In one embodiment, HBM™Hepatocyte Basal Medium(Lonza) supplemented with 10% Knockout™ serum replacement (KSR : ThermoFisher Scientific), 10 ng/ml TGFβ2 (Peprotech) and 25 ng/ml EGF (R&D) may be used.

In the step of differentiating hepatoblasts into cholangiocyte progenitors, the culturing conditions are not specifically limited. The cells may be cultured at about 30-40° C. and preferably about 37°C. under a CO₂-containing air atmosphere, and the concentration of CO₂ is preferably from about 2% to about 5%. During the culture, the medium may preferably be changed every day.

According to the method provided by this application, hepatoblasts are differentiated into cholangiocyte progenitors. The differentiation of hepatoblasts into cholangiocyte progenitors progresses with time from the DP stage to the RDP stage.

From non-patent literature 1, the present inventors noticed that AQP1 was not expressed in hepatoblasts and in the early DP stage but started to be expressed from the RDP stage, and considered that AQP1 could be a marker gene of cholangiocyte progenitors at the RDP stage of the differentiation. When hepatoblasts were differentiated into cholangiocyte progenitors by the method of the present application, cells on Day 3 expressed CK19 and SOX9 but not AQP1. This expression profile is similar to that of fetal cholangiocyte progenitors at around gestation week 8. In addition, cells on Day 7 of differentiation expressed AQP1 in addition to CK19 and SOX9. This expression profile is similar to that of fetal cholangiocyte progenitors at around gestation week 12-20. From these results, in the differentiation from the hepatoblasts into cholangiocyte progenitors, the cells expressing CK19 and SOX9 but not AQP1 may be identified as cells corresponding to those at the DP stage and the cells expressing CK19, SOX9 and AQP1 may be identified as cells corresponding to those at the RDP stage.

According to the present application, a method for identifying cholangiocyte progenitors that are at a stage corresponding to the RDP or later stage, and a method for isolating the identified cells are provided. In order to determine whether the cells are cholangiocyte progenitors at the RDP or later stage, expression of AQP1 gene may be used as an index. For example, human tissue or cultured human cells may be immuno-stained to determine whether the cholangiocyte progenitors are at the DP stage, or at the RDP or later stage. In addition, cells at the RDP or later stage and cells at DP stage can be separated by using the expression of AQP1 gene as an index.

In one embodiment of the method provided herein, the hepatoblasts may be those induced from mammalian pluripotent stem cells. In the specification and claims of the present application, pluripotent stem cells refer to stem cells which have pluripotency, that is the ability of cells to differentiate into all types of the cells in the living body, as well as proliferative capacity. Examples of the pluripotent stem cells include embryonic stem (ES) cells (J. A. Thomson et al., (1998), Science 282: 1145-1147; J. A. Thomson et al., (1995), Proc. Natl. Acad. Sci. USA, 92: 7844-7848; J. A. Thomson et al., (1996), Biol. Reprod., 55: 254-259; J. A. Thomson and V. S. Marshall (1998), Curr. Top. Dev. Biol., 38: 133-165), nuclear transfer embryonic stem (ntES) cells that can be obtained by nuclear transplantation into the ES cells(T. Wakayama et al., (2001), Science, 292: 740-743; S. Wakayama et al., (2005), Biol. Reprod., 72: 932-936; J. Byrne et al., (2007), Nature, 450: 497-502), germline stem cells (“GS cells”) (M. Kanatsu-Shinohara et al., (2003) Biol. Reprod., 69: 612-616; K. Shinohara et al., (2004), Cell, 119: 1001-1012), embryonic germ cells (“EG cells”) (Y. Matsui et al., (1992), Cell, 70: 841-847; J. L. Resnick et al., (1992), Nature, 359: 550-551), induced pluripotent stem (iPS) cells (K. Takahashi and S. Yamanaka (2006) Cell, 126: 663-676; K. Takahashi et al., (2007), Cell, 131:861-872; J. Yu et al., (2007), Science, 318: 1917-1920; Nakagawa, M. et al., Nat. Biotechnol. 26: 101-106 (2008); WO 2007/069666), pluripotent cells derived from cultured fibroblasts and bone marrow stem cells (Multi-lineage differentiating Stress Enduring cells. Muse cells) (WO 2011/007900). Preferably, the pluripotent stem cells are human pluripotent stem cells such as human ES cells and human iPS cells. Human iPS cells are particularly preferable.

Pluripotent stem cells may be those generated by a known method or commercially available cells. Pluripotent stem cells stocked for research or transplantation purpose with the information of the individual from which they were derived may also be used. A project to construct a versatile iPS cell bank is now in progress in Japan by using a human having a frequent HLA haplotype in homozygous as the donor (CYRANOSKI, Nature vol. 488, 139(2012)). Pluripotent stem cells that are obtained from an iPS cell bank as above may also be used.

Pluripotent stem cells used in this method may be substantially separated or dissociated into single cells by any method and the single cell suspension may be subjected to the culture. Alternatively, cell aggregation in which cells are attached each other nay be subjected to the culture. In order to obtain single cell suspension, cells in the pluripotent stem cell culture may be separated by, for example, mechanical separation or separation using a separation solution having protease and collagenase activities such as Accutase™ and Accumax™ (Innovative Cell Technologies, Inc.) that are solutions containing trypsin and collagenase, or a separation solution having only collagenase activity. Pluripotent stem cells may be cultured under adherent culture conditions in coated culture dishes.

Procedures for inducing hepatoblasts from pluripotent stem cells have been disclosed previously. For example, a method disclosed in WO2001/081549, WO2016/104717, Takayama K, et al, Stem Cell Reports. 1:322-335, 2013., Kajiwara M, et al., Proc Natl Acad Sci USA. 109: 12538-12543, 2012 and Hay DC et al., Stem Cells. 26: 894-902, 2008 may be employed.

In order to induce hepatoblasts from pluripotent stem cells, endoderm cells are first induced from the pluripotent stem cells, then, hepatoblasts are induced from endoderm cells. In one embodiment, pluripotent stem cells are cultured in a medium containing an activator of activin receptor-like kinase-4,7, a GSK3 inhibitor and a ROCK inhibitor to obtain endodermal cells. Endodermal cells may then be cultured in a medium containing BMP4 and FGF2 to obtain hepatoblasts.

The activator of activin receptor-like kinase-4,7 is a substance that activates ALK-4 and/or ALK-7. An activin is preferable and activin A is more preferable. GSK3 inhibitor is defined as a substance that inhibits the kinase activity of GSK-3β, for example, a substance that inhibits phosphorylation of β-catenin. Many GSK3 inhibitors have been known such as CHIR99021 and may be selected a suitable one. ROCK inhibitor may be any substance as long as the substance suppresses the Rho-kinase (ROCK) activity. Examples of ROCK inhibitors may include Y-27632.

The present application further provides a method for obtaining a cell culture having a bile duct-like 3D duct-like structure from hepatoblasts. In said method, hepatoblasts or cholangiocyte progenitors at a stage corresponding to the DP stage or the RDP stage that are induced from hepatoblasts may be cultured further in the presence of a 3D scaffold material in a medium comprising hepatocyte growth factor (HGF), EGF, a Notch signal ligand and a GSK3 inhibitor.

Various 3D scaffold materials for forming 3D structures of cultured cells have been known and are commercially available. The 3D scaffold materials used in the present method are not specifically limited. For example, polymer materials such as collagen based materials, polycaprolactone, polyglycolic acid, or a combination thereof may be used. The structure of the 3D scaffold material is not specifically limited and may be spongy structure. The 3D scaffold material may also be a material made from a biological material such as extracellular matrix or basement membrane matrix. Examples of 3D scaffold materials made from a biological material may include Matrigel™ (BD Biosciences) , Type I collagen gel and Type TV collagen gel.

Matrigel™ basement membrane matrix is a soluble basement membrane preparation extracted from Engelbreth-Holm-Swarm (EHS) mouse sarcoma, which is rich in extracellular matrix proteins and is mainly composed of laminin, collagen IV, entactin, and heparan sulfate proteoglycans. It also contains other growth factors such as TGFβ, fibroblast growth factor, tissue plasminogen activator, and EHS.

In one embodiment, a gel formed by mixing type I collagen and Matrigel™ is exemplified as the 3D scaffold material. In a specific embodiment, the gel is prepared by mixing 60% type I collagen and 40%. Matrigel™ basement membrane matrix with reduced growth factors. Hepatoblasts or cholangiocyte progenitors may be encapsulated in the 3D scaffold by mixing the gel with the cells and allowing the mixture to solidify.

Cells encapsulated in the 3D scaffold material are cultured in a medium supplemented with HGF, EGF, a Notch signal ligand, and a GSK3 inhibitor. Hepatocyte growth factor (HGF) and epidermal growth factor (EGF) are commercially available. Human HGF and EGF are suitably used when human hepatoblasts are used as the starting material. GSK3 inhibitor may be any of the known substances and CHIR 99021 is exemplified. As ligands for Notch signal, Delta like signals such as Delta Like Protein 1, Delta Like Protein 3 and Delta Like Protein 4, and Jagged ligands such as Jagged-2 and Jagged-1 are exemplified. Jagged-1 (JAG1) may preferably be used.

The amount of each component added to the medium may be appropriately determined. The concentration of HGF in the medium may be 2-200 ng/ml, preferably 4-100 ng/ml, and for example about 20 ng/ml. The concentration of EGF in the medium may be 5-500 ng/ml, preferably 10-250 ng/ml, and for example, about 50 ng/ml. When JAG1 is used as a Notch signal ligand, the concentration of JAG1 in the medium may be 5-500 ng/ml, preferably 10-250 ng/ml, and for example about 50 ng/ml. When CHIR 99021 is used as a GSK3β inhibitor, the concentration in the medium is 0.3-30 μM, preferably 0.6-15 μM, and for example about 3 μM.

In one embodiment, HBM™ (Hepatocyte Basal Medium, Lonza) supplemented with 10% KnockOut™ serum replacement (KSR : ThermoFisher Scientific), 20 ng/ml HGF (Peprotech), 50 ng/ml EGF (R&D), 50 ng/ml Jagged-1 R&D) and 3 μM CHIR99021 (StemRD) may be used.

Preferably, the cells may be cultured using a culture vessel equipped with a culture insert. The cells encapsulated in the 3D scaffold material may be placed on the culture insert, and the culture medium is added into the culture insert and the well under it, respectively. The cells may be cultured at a temperature about 30-40° C., preferably about 37° C. under a CO₂-containing air atmosphere, but not limited to such conditions. The concentration of CO₂ in the air may preferably be about 2-5%.

The medium may preferably be changed every 2 days during the culture. The culture period is not particularly limited, and may be from 8 to 15 days, and for example about 10 days, regardless of the cell types subjected to the 3D culture.

According to the method provided herein, it has become possible to induce hepatoblasts into DP-like cholangiocyte progenitors and then, RDP-like cholangiocyte progenitors in a stepwise manner.

### EXAMPLE

Establishment of human iPS ceils

Establishment of human iPS cell line 23C27

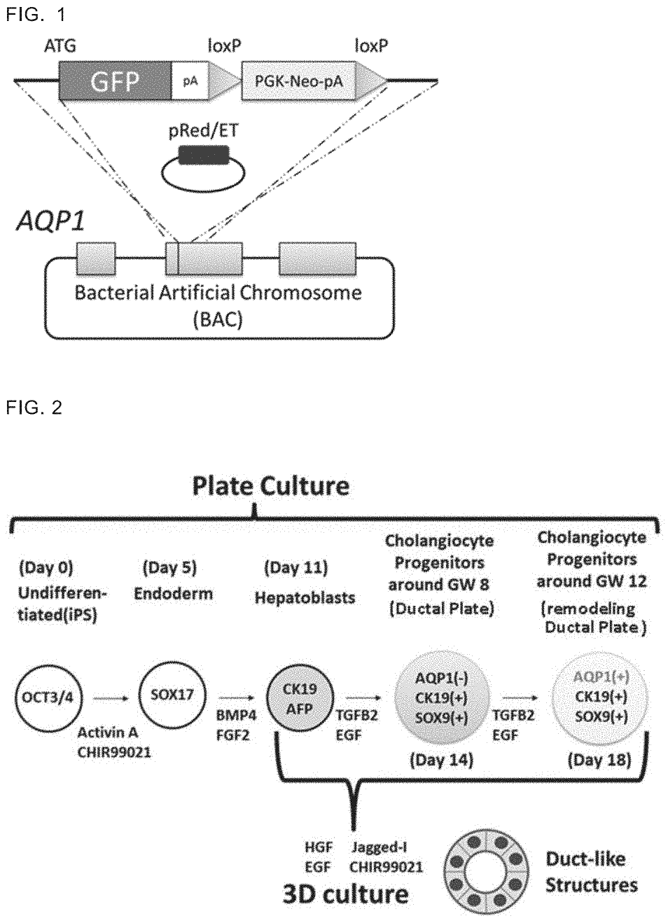

An AQP1-GFP construct was prepared by introducing the base sequence of GFP-PGK-Neo into an E-coli in which human AQP1 base sequence had been introduced (Bacterial Artificial Chromosome: Children's Hospital Oakland Research Institute). A schematic diagram of the AQP1-GFP construct is shown in FIG. 1.

The AQP1-GFP construct was introduced into cells of human iPS cell line 585A1 (Center for iPS Cell Research and Application, Kyoto University) by means of electroporation and AQP1-GFP reporter human iPS cell line 23C27 was obtained. Cholangiocyte progenitors were induced from thus obtained iPS cells. Protocols for inducing cholangiocyte progenitors are summarized in FIG. 2.

(1) Differentiation of human iPS cells into endoderm cells 23C27 human iPS cells grown to approximately 80% confluent were used. The cultured and undifferentiated human iPS cells were seeded at a cell density of 7.7×10⁴ cells/cm²(3.0×10⁵ cells/3.9 cm²) (Day 0). The used medium was RPMI1640 (Nacalai Tesque) supplemented with 1×B27 supplement (Thermo Fisher Scientific), 1×peniciline/streptomycin (ThermoFisher Scientific), 100 ng/ml activin A (R&D), 1-3 μM CHIR99021 (StemRD; Day 0: 3 μM, Day 1-3: 1 μM and Day 3-5: 0 μM,) and 10 μM y-27632 (Wako; Day 0: 10 μM, Day 1-5: 0 μM).

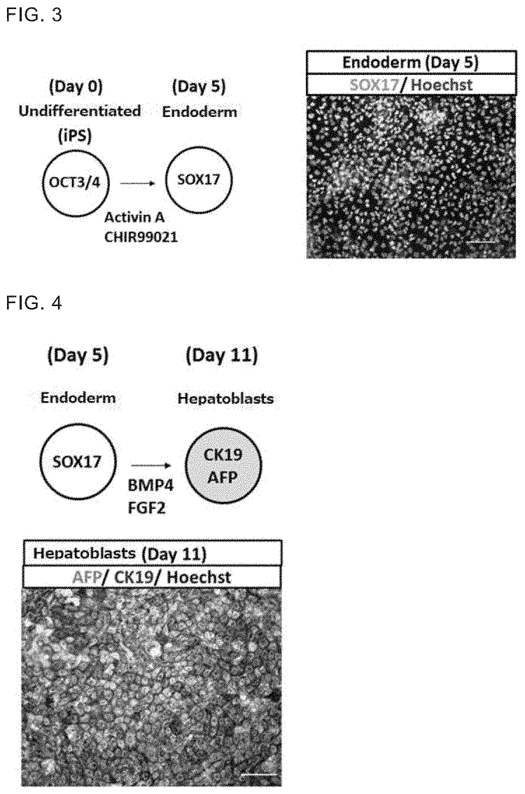

The cells were cultured for 5 days. During the culture, the medium was changed with freshly prepared medium every day. CHIR99021 concentrations in the medium were adjusted to 1 μM on Day 1 and 2, to 0 μM on Day 3 and Day 4. Y-27632 concentrations were adjusted to 0 μM on Day 1 and thereafter. Thus obtained culture cells were fixed by a conventional method and immunostained to observe the expression of sox17. Hoechst 33342 was used for nuclear staining. Result is shown in FIG. 3. The obtained cells were positive for SOX17 and confirmed that the iPS cells were differentiated into endoderm cells.

(2) Differentiation of endoderm cells into hepatoblasts The medium of the endoderm cell culture (Day 5) obtained in step (1) was changed with a medium for hepatoblasts induction. As the hepatoblasts induction medium, KnockOut™ DMEM (KODMEM; ThermoFisher Scientific) supplemented with 10% KnockOut™ serum replacement (KSR: ThermoFisher Scientific), 1 mM L-glutamine (ThermoFisher Scientific), 1% (vol/vol) non-essential amino acids (ThermoFisher Scientific), 1×peniciline/streptomycin (ThermoFisher Scientific), 0.1 mM 2-mercapt ethanol (ThermoFisher Scientific), 1% (vol/vol) DMSO (Sigma), 20 ng/ml BMP4 (Peprotech) and 10 ng/ml FGF2 (Wako) was used.

The cells were cultured for additional 6 days (Day 5-11). During the culture, the medium was changed every day. A part of the obtained cells were fixed by a conventional method and immunostained to observe the expression of markers CK19 and AFP respectively. Hoechst 33342 was used for nuclear staining. Result is shown in FIG. 4. The obtained cells were positive for CK19 and AFP, and were confirmed to be hepatoblasts.

(3) Differentiation into cholangiocyte progenitors Hepatoblast cell culture on Day 11 obtained in step (2) were differentiated into cholangiocyte progenitors. As the cholangiocyte progenitors induction medium, HBM™ Hepatocyte Basal Medium (Lonza) supplemented with 10% Knockout™ serum replacement (KSR; ThermoFisher Scientific), 10 ng/ml TGFβ2 (Peprotech) and 25 ng/ml EGF (R&D) was used.

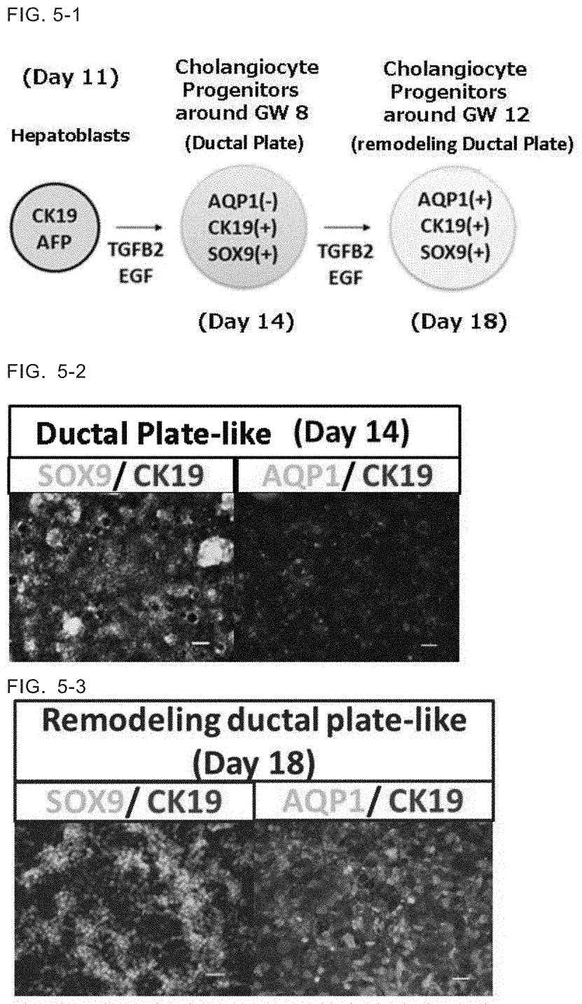

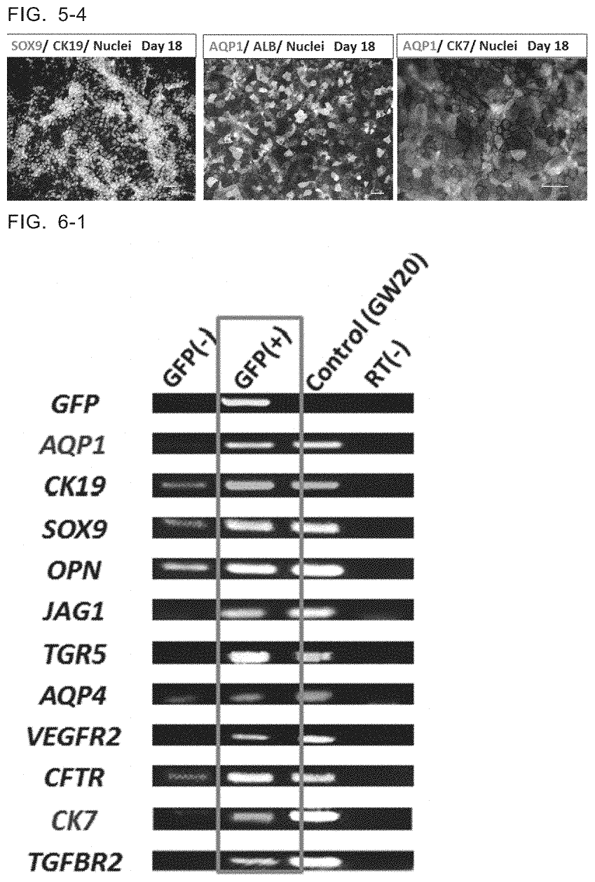

The cells were cultured for additional 7 days (Day 11-18). During the culture, the medium was changed with freshly prepared medium every day. After three days culture in step (3) (on Day 14), a part of the cultured cells were obtained and fixed by a conventional method. Expression of markers specific for cholangiocyte progenitors were confirmed by immunostaining. Results are shown in FIG. 5-2. The obtained cultured cells were negative for APQ1. Since APQ1 is expressed at around gestation week 12-16, this result suggests that the cells on Day 14 of differentiation correspond cells before GW 12-16. Many of the cells were positive for SOX9, the gene which is expressed by gestation week 6-8. According to those results, cells on Day 14 of differentiation had a gene expressing profile comparative to that of cholangiocyte progenitors at the ductal plate stage around gestation week 8.

The cells were cultured under the same conditions for 7 days in total in step (3) (until Day 18 of differentiation). Thus obtained cells were subjected to immunostaining similarly. Results are shown in FIG. 5-3. Many of the obtained cells were positive for AQP1, the gene which is expressed around gestation week 12-16. The cells on Day 18 were subjected to more precise immunostaining. Result are shown in FIG. 5-4. The cells on Day 18 were positive for SOX9, CK19, AQP1 and CK7, and confirmed to be corresponding to cholangiocyte progenitors at the remodeling ductal plate stage at around gestation week 12.

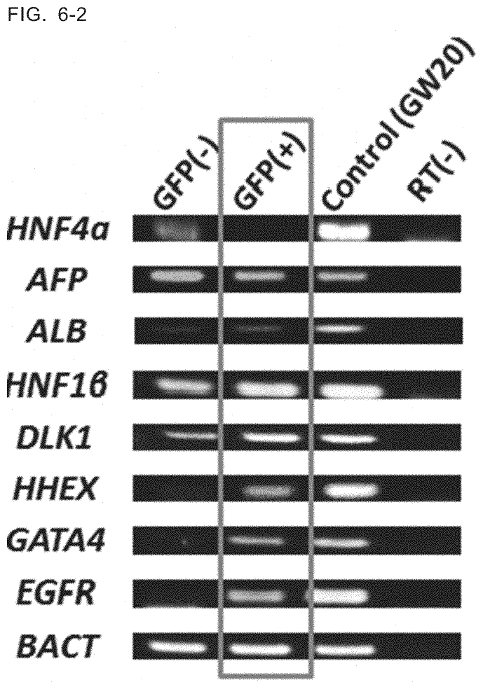

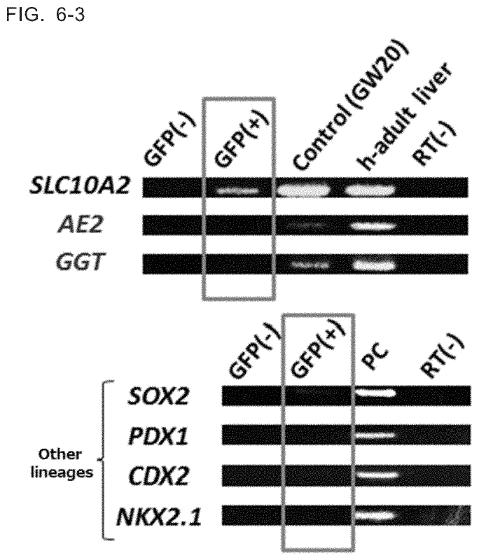

(4) Confirmation of cholangiocyte progenitors AQP1 positive and AQP1 negative cells were isolated respectively from the cell population at Day 18 obtained in step (3) by isolating GFP positive cells using a flow cytometer. Each cell population was examined by PCR for marker genes that have been known to be expressed in cholangiocyte progenitors at around gestation weeks 12-20. As a control, gestation week 20 fetal liver was similarly examined for the expression of each gene by PCR. In addition, the expression of some marker genes was also examined in adult liver having cholangiocytes. The expression of genes known as marker genes specific for brain, pancreas, colon and trachea in the cells were also examined by PCR. Results are shown in FIGS. 6-1 to 6-3. It was confirmed that the cholangiocyte progenitors obtained by the method of this application showed the gene expression profile similar to the cholangiocyte progenitors at the remodeling ductal plate stage of around GW 12-20. On the other hand, none of the genes specific for the other organs was expressed in the cells (FIG. 6-3).

(5) Formation of the 3D duct-like structure from hepatoblasts and cholangiocyte progenitors

The hepatoblasts (Day 11) obtained in step (2) and cholangiocyte progenitors (Day 14) obtained in step (3) were subjected to the 3D culture. 3D scaffold was made of the following materials:

BD Matrigel™ basement membrane matrix growth factor reduced, 10 ml vial (BD354230),
Collagen Type I, rat tail (ThermoFisher Scientific A104830), Cell Culture Insert Companion Plate (BD falcon 353504),
Culture Inserts for 24 well plates, 1.0 μm, PET, transparent (FALCON #353104).

Collagen type I and 40% matrigel™ were mixed to prepare a gel. The Day 11 and Day 14 cells were independently mixed with the gel so that the cell density in the gel was 1.0×10⁶/100 μL. 100 μL/well of the gel containing the cells was loaded in the culture insert and incubated at 37° C. for 2 hours to solidify the gel. After the solidification, 200 μl and 500 μl of culture medium was added into the insert and the well under it, respectively. The cells were cultured for 10 days. The culture medium was changed every two days.

HBM™ Hepatocyte Basal Medium (Lonza) supplemented with 10% KnockOut™ serum replacement (KSR; ThermoFisher Scientific), 20 ng/ml HGF (Peprotech), 50 ng/ml EGF (R&D), 50 ng/ml Jagged-1 (R&D) and 3 μM CHIR99021 (StemRD) was used.

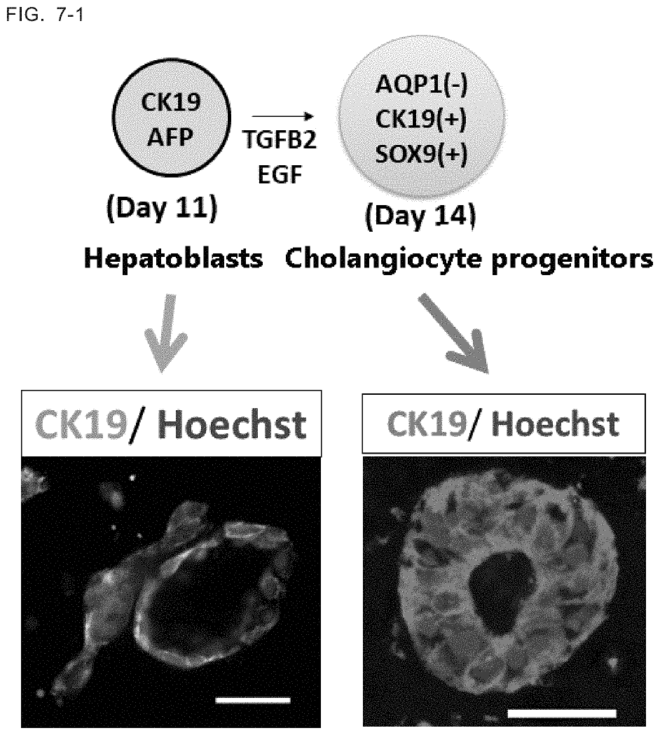

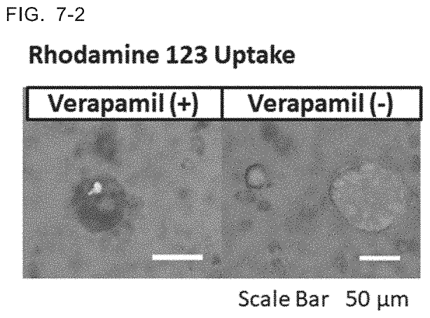

After the 10 days culture, the 3D scaffold material in which the cells were encapsulated and cultured were taken out and fixed by paraformaldehyde (PFA) and obtained sections were examined for the expression of CK19 by immunostaining. Hoechst 33342 was used for nuclear staining. Results are shown in FIG. 7-1. Both Day 11 and Day 14 cells subjected to the 3D culture formed bile duct-like 3D duct-like structure. Cells of Day 18 of differentiation were subjected to the 3D culture in the same manner as above. As a result, 3D duct-like structure was formed.

To evaluate the function of the obtained cell culture having the 3D duct-like structures, rhodamine 123 transportation assay was conducted.

Reagents used here were as follows:

### Rhodamine 123: Sigma-Aldrich, Cat#R8004

### Verapamil: Sigma-Aldrich, Cat#V106-5MG

The cell culture having the 3D duct-like structure was incubated in 100 μl Rhodamine 123 solution in the presence or absence of 20 μM verapamil for 10 minutes. Rhodamine 123 uptake was observed under a fluorescence microscopy. Results are shown in FIG. 7-2.

Rhodamine 123 is taken up by a transporter called MDR1/p-glycoprotein expressed in the bile duct epithelium. Verapamil is an inhibitor of MDR1/p-glycoprotein. It was confirmed that the cell culture having the 3D duct-like structure took up rhodamine 123, and that the rhodamine 123 uptake was remarkably suppressed in the presence of verapamil. From this result, it was confirmed that the culture having the obtained 3D duct-like structure had the physiological function as a bile duct.

1. A method for generating cholangiocyte progenitors, comprising the steps of:
providing hepatoblasts, and
culturing the hepatoblasts in a medium comprising TGFβ and EGF.

2. The method according to claim 1, wherein TGFβ is TGFβ2.

3. The method according to claim 1, wherein the cholangiocyte progenitors to be generated are positive for CK19 and SOX9, and negative for AQP1.

4. The method according to claim 1, wherein the cholangiocyte progenitors to be generated are ductal plate-like cholangiocyte progenitors.

5. The method according to claim 1, wherein the cholangiocyte progenitors to be generated are positive for all of CK19, SOX9, and AQP1.

6. The method according to claim 1, wherein the cholangiocyte progenitors to be generated are remodeling ductal plate-like cholangiocyte progenitors.

7. The method according to claim 1, wherein the method further comprises a step of inducing hepatoblasts from pluripotent stem cells.

8. The method according to claim 7, wherein the pluripotent stem cells are human cells.

9. The method according to claim 8, wherein the pluripotent stem cells are human ES cells or human iPS cells.

10. A cholangiocyte progenitor cell culture, which was generated by the method according to claim 1.

11. The cholangiocyte progenitor cell culture of claim 10, wherein the cholangiocyte progenitors are ductal plate-like cholangiocyte progenitors.

12. The cholangiocyte progenitor cell culture of claim 10, wherein the cholangiocyte progenitors are remodeling ductal plate-like cholangiocyte progenitors.

13. A method for generating a three-dimensional duct-like structure of cholangiocyte progenitors, which comprises the steps of:
providing hepatoblasts or cholangiocyte progenitors, and
culturing the hepatoblasts or cholangiocyte progenitors in a medium comprising HGF, EGF, a Notch inhibitor and a GSK3 inhibitor in the presence of a three-dimensional scaffold material.

14. A three-dimensional duct-like structure of cholangiocyte progenitors, which is generated by a method comprising the steps of:
providing hepatoblasts or cholangiocyte progenitors, and
culturing the hepatoblasts or cholangiocyte progenitors in a medium comprising HGF, EGF, a Notch inhibitor and a GSK3 inhibitor in the presence of a three-dimensional scaffold material.
